(19) United States
(12) Patent Application Publication
(10) Pub. No.: US 2007/0078401 A1
(43) Pub. Date: Apr. 5, 2007

(54) Intravenous tubing protector and support system

(76) Inventor: Susan J. Servoss, Niantic, CT (US)

Correspondence Address: LAW OFFICE OF STEVEN B. LEAVITT, L.L.P., P.O. BOX 537, ROWLETT, TX, 75030-0537, US

(21) Appl. No.: 11/200,733

(22) Filed: Aug. 10, 2005

(51) Int. Cl. A61M 5/32

(52) U.S. Cl. 604/179

(57) ABSTRACT

The present invention relates to an intravenous tubing protector or organizing wrap support designed to consolidate multiple numbers of tubing by encircling all the tubing together. More particularly, the device would organize and prevent the tangling of intravenous tubing and may be made up of a flat, elongate, flexible and elastic strip of material of sufficient length to encircle at least two or more numbers intravenous tubing or may be made of a fitted and potentially predetermined width and length of semi-formed material that would retain its shape around the tubing but yet be comfortable to the patient. Attached to the ends of the flexible material that may be overlapped would likely include one or more fastener assemblies constructed of respective engagable upper and lower respective pluralities of resilient engaging elements which are separated by a distance sufficient to ensure that the captured loop of the intravenous tubing remains unkinked.

### BACKGROUND OF THE INVENTION

1. Technical Field

The present invention relates to intravenous (IV) therapy and administration of liquid nutrients and/or medications to a patient. More particularly, the present invention relates to the administration of two or more intravenous tubes with the utilization of a flexible insulate, cover, or wrapping, or form fitted wrap for the organization, protection and consolidation of multiple intravenous tubes to prevent the tubes from being tangled, disorganized and potentially pulled out of a patient.

2. Description of Related Art

The administration of liquid nutrients and medications via intravenous tubes is well known in the art and are typically used when a patient may require one or more medications and/or liquid nutrients at a time. Generally, the liquid nutrients or medical solutions are delivered into the patient's bloodstream directly from an intravenous fluid line. Intravenous fluid lines generally consist of flexible plastic tubing. In a typical intravenous fluid administration system, the intravenous fluid line connects directly with the source of the intravenous fluid. The fluid source is commonly an elevated intravenous fluid storage container made of a flexible wall plastic construction. In such a typical system, the flow of intravenous fluid to the patient is gravity driven.

It is not uncommon for a plurality of intravenous fluid lines, each connected to a different source of intravenous fluid, to simultaneously be used to deliver intravenous fluid therapy to a single patient. The simultaneous use of several different intravenous fluid lines, sometimes ten or more wherein each line is connected to a different source of intravenous fluid, is not uncommon in modem medical practice.

The simultaneous use of multiple intravenous fluid lines has led to some major problems in the current art of intravenous fluid administration, including problems with tubing course and distinction among multiple tubes. Further, the advances that have been made toward solving these problems have been severely limited by an inability to match utility with quality control. Additional problems include the potentially dangerous disruptions in fluid flow resulting from kinks, tangles, or the like in the intravenous tubing. Such kinks create physical breaks in the continuity of the fluid communication between the patient's bloodstream and the source of the intravenous fluid. Similar problems may arise as a result of having to move the patient or having the patient sitting or lying on one or more of the tubing. All the above problems could further result in danger to the patient as a result of the tubing being pulled out or as a result of the flow of the nutrients or medications being blocked.

Also, quality control is a necessary and vital component of the administration of intravenous fluids. Two critical means in which the health care provider monitors the quality of the intravenous fluid administration are to visually examine the fluid in the intravenous fluid container and to visually examine the fluid in the intravenous tubing as it enters the patient's body. By casually monitoring the course of the infusion, the medical care provider can be certain that the quality of the intravenous fluid is satisfactory and that the process of the administration is progressing as desired. For example, visual monitoring of the intravenous tubing near the injection site can detect the backing of blood into the intravenous tubing which would indicate an indication of a troubled intravenous fluid administration. Also visual monitoring of the intravenous fluid reservoir can check for clarity of the fluid, the presence of contaminants, precipitation, and so forth.

Therefore, an unobstructed view of the fluid in both the intravenous tubing near the patient's body and in the intravenous fluid reservoir is critical to proper quality control in intravenous fluid administration. Any intravenous fluid administration that uses indicia which block an unobstructed view of the intravenous fluid at either or both the intravenous tubing near the patient's body and/or the intravenous fluid container suffers from a defect in quality control. In addition, tangled tubing, or tubing that is under the patient will not as likely be notice if there are multiple lines being utilized.

The most commonly used method of securing injection cannula, infusion tubes and other tubular items of equipment during intravenous injections, has been to strap such items to the patient with ordinary adhesive tape. This has proven unsatisfactory because the tape must be removed each time the held item is changed, causing discomfort to the patient and possible skin damage. Moreover, the items are usually not held sufficiently secured and are likely to move relative to the patient each time the patient moves, again causing pain and possible injury, or even complete dislodgement of the item.

One prior art tubing support system in a related field is that found in U.S. Pat. No. 3,726,280 to Lacont for a “Catheter Support” in which en elongated elastic band is used to encircle the limb and then to double back upon itself to capture the catheter tubes by a system of VELCRO (a Registered Trademark of Velcro Corporation, New York, N.Y.)-type hook and loop fasteners.

Another prior art supporting system is that found in U.S. Pat. No. 3,834,380 to Boyd for a “Holder For Intravenous Injection Cannula and Tubing” which discloses a holder comprising a strip of adhesively backed tape to which is attached a longitudinally split clamping tube of flexible plastic material which receives the intravenous tube and over which a flap of hook and loop fastener is secured to retain the tubing within the clamping tube.

Also, U.S. Pat. No. 4,671,787 to Widman describes a “wrap” that is made up of a length of material sufficient to surround the limb at the injection site and to additionally overlap itself to at least 15% of its length. The two ends of the captured intravenous tubing loop are first captured by two flaps of material having respective pluralities of resilient engaging elements for releasably securing the flaps, located at one end of the wrap. The length of material is then wrapped around the limb to overlap the end which has captured the intravenous loop and then to resecure the wrap to itself by a third fastener of resilient elements.

### SUMMARY OF THE INVENTION

The present invention relates to an intravenous tubing protector and/or form fitted organizing wrap support designed to consolidate multiple numbers of tubing by encircling all the tubing together. The device would prevent the tangling of the tubing and prevent the need to continually replace tubing that had been pulled out as a result of the tubing being pulled and tangled together or from the patient's movement. The intravenous tubing protector system of this invention may be made up of a flat, elongate, flexible and elastic strip of material of sufficient length to encircle at least two or more intravenous tubing. However, the tubing protector may be made of a fitted and potentially predetermined width and length of semi-formed material that would retain its shape around the tubing but yet be comfortable to the patient. Attached to the ends of the flexible material that may be overlapped would likely include one or more fastener assemblies constructed of respective engagable upper and lower respective pluralities of resilient engaging elements which are separated by a distance sufficient to ensure that the captured loop of the intravenous tubing remains unkinked. The respective engaging element(s) of the fastener would allow for the capturing the intravenous tubing and be attached to the extended portion of the fastener element by an additional respective fastener element on the overlapping end.

### BRIEF DESCRIPTION OF THE DRAWINGS

The novel features believed characteristic of the invention are set forth in the appended claims. The invention itself, however, as well as a preferred mode of use, further objectives and advantages thereof, will be best understood by reference to the following detailed description of illustrative embodiments when read in conjunction with the accompanying drawings, wherein:

FIG. 1 is a perspective view of the tubing protector made in accordance with an embodiment of the present invention;

FIG. 2 is a perspective view of an alternate embodiment of the present invention; and

FIG. 3 is a perspective view of an alternate embodiment of the present invention.

### DETAILED DESCRIPTION

The tubing protector of the present invention is designed to support intravenous tubing for intravenous therapy procedures. The device serves to secure the tubing from being tangled, kinked or pulled out of the patient and is capable of encircling a variety of different numbers of tubing; however, it is envisioned that several different standard sizes of the tubing protector be employed to handle respective numbers and lengths of tubing. In addition, the tubing protector is designed to capture multiple intravenous tubing and consolidate the tubing together to prevent tangling and a potential kink that would thereby prevent the continued flow of the therapeutic liquid into the blood vessel of the patient.

This tubing protector may be designed to be employed in most usages with Velcro® type hook and loop fasteners or any other type of button, snap or similar fastening device or means. Velcro®-type hook and loop fasteners are especially suitable for medical applications because they are washable and flexible as well as easily applied and removed. Additional fastener means such as pronged metal clips may also be employed in conjunction with the tubing protector of this invention to reinforce the holding power of the Velcro® fasteners. The significant consideration is that the flaps at the one end of the tubing protector are securely fastened down to the underlying material of the tubing protector itself such that the intravenous tubing is securely confined beneath the flaps of the first and second fastener elements.

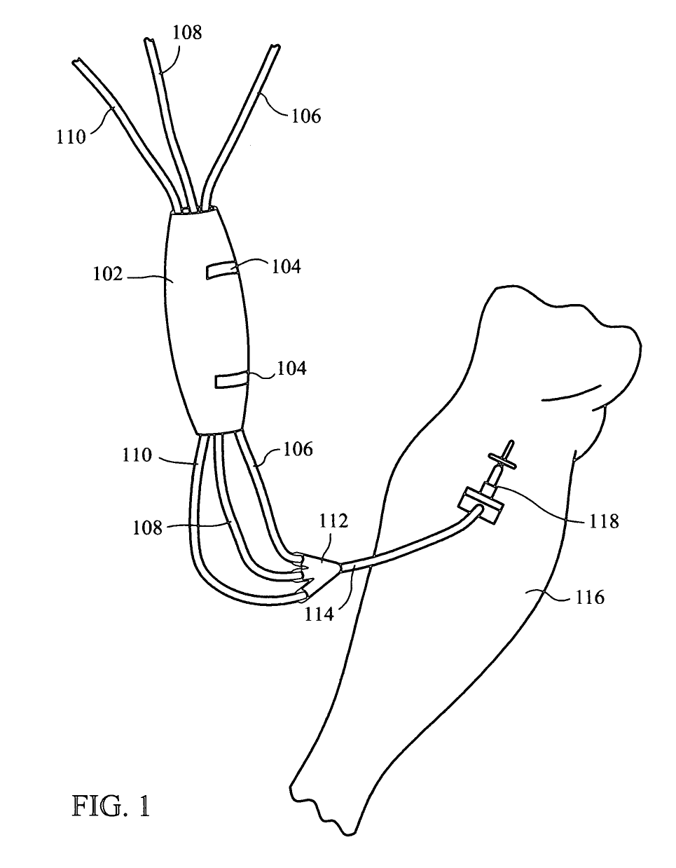

FIG. 1 is a perspective view of tubing protector 102 encircling three intravenous tubes 106, 108, and 110. Preferably, intravenous tubes 106, 108, and 110 are clear flexible plastic tubes but may be any tubing capable of carrying fluids. Tubing protector 102 encircles intravenous tubes 106, 108, and 110 and is secured with one or more fastener 104. Fastener 104 may be hook an loop fastener such as Velcro®, snaps, buttons, adhesive, or other similar attachment means. Tubing protector 102 prevents the tangling of the tubing and prevent the need to continually replace tubing that had been pulled out as a result of the tubing being pulled and tangled together or from the patient's movement.

Intravenous tubes 106, 108, and 110, are all connected to tube joiner 112 which is connected to main intravenous tube 114. Main intravenous tube 114 is connected to at least one intravenous needle 118 in arm 116 of a recipient of intravenous fluid. If more than one intravenous needle 118 is used, then main intravenous tube would split to correspond to the number of intravenous needles 118 used.

Tube joiner 112 may be clear and is preferably made of plastic, teflon, or some other similar material. While three intravenous tubes are shown, the present invention can accommodate any number of intravenous tubes. For example, if six intravenous tubes are used, then tub joiner 112 would have six openings or ports to receive the six intravenous tubes. Tub joiner 112 may have caps on the openings or ports. The caps would be removed if the opening or port was used. Therefore, if the tub joiner could accommodate six intravenous tubes but only three intravenous tubes are uses, then the remaining openings or ports would be caped so as not to be exposed and open.

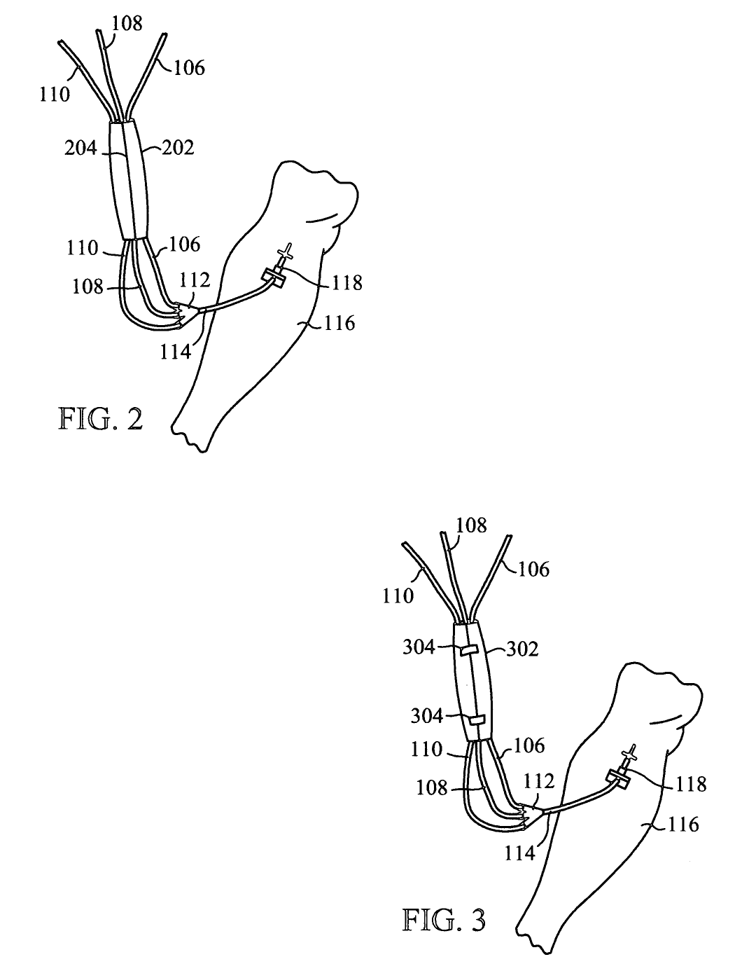

FIG. 2 shows formed tubing protector 202 having a slit 204, wherein the tubing protector comprises a flexible rubber, foam or similar type hose material. Tubing protector 202 may be preformed such that would retain its shape but allow for some flexibility in separating slit 204 to allow for insertion of the tubing. Slit 202 could be easily separated to facilitate inserting a tubing but would naturally return to a closed position enveloping intravenous tubes 106, 108, and 110. Tubing protector 202 prevents the tangling of the tubing and prevent the need to continually replace tubing that had been pulled out as a result of the tubing being pulled and tangled together or from the patient's movement.

Intravenous tubes 106, 108, and 110, are all connected to tube joiner 112 which is connected to main intravenous tube 114. Main intravenous tube 114 is connected to at least one intravenous needle 118 in arm 116 of a recipient of intravenous fluid.

FIG. 3 shows flexible tubing protector 302 having fasteners 304. Flexible tubing protector 302 is a flat, elongate, flexible elastic strip of material. The material may be woven or non-woven material and may be synthetic. In use, the flexible tubing protector 302 folds over onto itself and may be attached with one or more Velcro® strips 304 such that flexible tubing protector 302 encircles intravenous tubes 106, 108, and 110 and is secured with one or more Velcro® fasteners 104. Tubing protector 302 prevents the tangling of the tubing and prevent the need to continually replace tubing that had been pulled out as a result of the tubing being pulled and tangled together or from the patient's movement.

Intravenous tubes 106, 108, and 110, are all connected to tube joiner 112 which is connected to main intravenous tube 114. Main intravenous tube 114 is connected to at least one intravenous needle 118 in arm 116 of a recipient of intravenous fluid.

It should be understood that the foregoing relates to exemplary embodiments of the invention and that modifications may be made without departing from the spirit and scope of the invention as set forth in the following claims.

What is claimed is:

1. An apparatus for the consolidation of at least two tubing, comprising:
at least two tubing having a first end and a second end;
a connector attached to the second end of each tubing wherein the connector has an inlet and an outlet and the inlet side has at least two ports for receiving the tubing; and
an outlet tube attached to the outlet end of the connector.

2. The apparatus of claim 1 further comprising:
a tubing protector of sufficient length to encircle the at least two tubing; and
at least one fastener assembly attached to the tubing protector.

3. The apparatus of claim 2, wherein the fastener assembly is constructed of engagable upper and lower hook and loop elements.

4. The apparatus of claim 3, wherein the fastener assembly is located proximate to an overlapped end and having a permanently closed hinge line perpendicular to the long axis of the material joining the upper and lower hook and loop elements.

5. The apparatus of claim 5, wherein the fastener assembly permanently attached to the material.

6. The apparatus of claim 2, wherein the fastener assembly is a hook and loop fastener.

7. The apparatus of claim 2, wherein the tubing protector is made of rubber.

8. The apparatus of claim 2, wherein the tubing protector is made of foam.

9. The apparatus of claim 2, wherein the tubing protector is made of fabric.

10. The apparatus of claim 9, wherein the fabric is woven material.

11. The apparatus of claim 1, wherein the tubing is intravenous tubing.

12. The apparatus of claim 1 wherein each port has a cap.

13. The apparatus of claim 1 wherein the connector is clear.

14. The apparatus of claim 1 wherein the outlet tube is clear.

15. The apparatus of claim 13 wherein the outlet tube is intravenous tubing.

16. The apparatus of claim 1 wherein the outlet tube is attached to an IV needle.

17. An apparatus for the consolidation of at least two tubing, comprising:
at least two tubing having a first end and a second end;
preformed housing of sufficient length to encircle the at least two tubing;
a slit in the tubing protector to allow for insertion of the tubing;
a connector attached to the second end of each tubing wherein the connector has an inlet and an outlet and the inlet side has at least two ports for receiving the tubing; and
an outlet tube attached to the outlet end of the connector.

18. The apparatus of claim 17 wherein the preformed housing is made of foam.

19. The apparatus of claim 17 wherein the preformed housing is made of rubber.

20. The apparatus of claim 17, wherein the tubing is intravenous tubing.
